(19) United States
(12) Patent Application Publication
(10) Pub. No.: US 2024/0206336 A1
(43) Pub. Date: Jun. 20, 2024

(54) COMPOUND

(71) Applicant: Sumitomo Chemical Co., Ltd., Tokyo (JP)

(72) Inventors: Michal Maciejczyk, Godmanchester (GB); Nir Yaacobi-Gross, Godmanchester (GB); Florence Bourcet, Cambridge (GB)

(73) Assignee: Sumitomo Chemical Co., Ltd., Tokyo (JP)

(21) Appl. No.: 18/099,857

(22) Filed: Jan. 20, 2023

(30) Foreign Application Priority Data
Nov. 28, 2022 (GB) 2217852.9

(51) Int. Cl. H10K 85/60; C07D 495/22

(52) U.S. Cl. CPC H10K 85/6576; C07D 495/22; H10K 85/626; H10K 85/655; H10K 30/30

(57) ABSTRACT

A compound of formula (I):

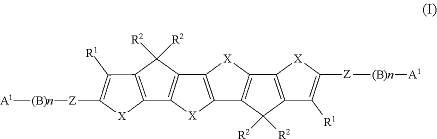

wherein: each X is independently O or S; R¹ in each occurrence is independently H a substituent; R² in each occurrence is independently a substituent; B in each occurrence is independently a bridging unit; n in each occurrence is independently 0 or 1; A¹ in each occurrence is independently an electron-accepting group; Z is a direct bond or, together with R¹, forms a monocyclic or fused ring; and the compound of formula (I) is substituted with at least one substituent of formula (II):

-(L)p-[(OCH₂CH₂)r-R³]q (II)

wherein: L is a linking group; p in each occurrence is independently is 0 or 1; r in each occurrence is independently an integer from 1 to 10; R³ is H or a substituent; and q is 1 if p is 0 and q is at least 1 if p is 1.

### RELATED APPLICATIONS

This application claims priority to United Kingdom Patent Application GB 2217852.9, filed Nov. 28, 2022, the contents of which are incorporated herein by reference.

### BACKGROUND

Embodiments of the present disclosure relate to electron-accepting compounds and more specifically compounds suitable for use as an electron-accepting material in a photoresponsive device.

An organic photodetector may contain a photoactive layer of a blend of an electron-donating material and an electron-accepting material between an anode and a cathode. Known electron-accepting materials include fullerenes and non-fullerene acceptors (NFAs).

Meng et al, “Replacing Alkyl with Oligo(ethylene glycol) as Side Chains of Conjugated Polymers for Close π-π Stacking”, Macromolecules 2015, 48, 13, 4357-4363 describes a series of conjugated polymers with oligo(ethylene glycol) (OEG) or alkyl chain as the side chain and poly[2,7-fluorene-alt-5,5-(4,7-di-2-thienyl-2,1,3-benzothiadiazole)] as the polymer backbone.

Zhang et al, “Effect of Replacing Alkyl Side Chains with Triethylene Glycols on Photovoltaic Properties of Easily Accessible Fluorene-Based Non-Fullerene Molecular Acceptors: Improve or Deteriorate?”, ACS Appl. Energy Mater. 2018, 1, 3, 1276-1285, describes non-fullerene acceptors for photovoltaic cells.

Chen et al, “A guest-assisted molecular-organization approach for >17% efficiency organic solar cells using environmentally friendly solvents” Nat. Energy 6, 1045-1053 (2021) describes compounds for use in organic solar cells.

Cui et al. “Designing Nonfullerene Acceptors with Oligo(Ethylene Glycol) Side Chains: Unraveling the Origin of Increased Open-Circuit Voltage and Balanced Charge Carrier Mobilities”, Chemistry, an Asian Journal, Volume 16, Issue 17, Sep. 1, 2021, 2481-2488 describes non-fullerene acceptors.

Jang et al, “A High Dielectric N-Type Small Molecular Acceptor Containing Oligoethyleneglycol Side-Chains for Organic Solar Cells”, Chinese Journal of Chemistry, Volume 36, Issue 3, March, 2018, 199-205 describes non-fullerene acceptors.

Chen et al, “Diketopyrrolopyrrole-based Conjugated Polymers Bearing Branched Oligo(Ethylene Glycol) Side Chains for Photovoltaic Devices”, Angewandte Chemie, Volume 55, Issue 35, Aug. 22, 2016, 10376-10380 describes polymers for use in photovoltaic devices.

Brebels et al, “An effective strategy to enhance the dielectric constant of organic semiconductors-CPDTTPD-based low bandgap polymers bearing oligo(ethylene glycol) side chains”, J. Mater. Chem. C, 2018, 6, 500-511 describes polymers for use in photovoltaic devices.

WO 2022/129137A1 describes organic compounds for use in organic photoresponsive devices.

WO 2022/033993A1 describes organic compounds for use in organic photoresponsive devices.

U.S. Pat. No. 11,005,043B2 describes organic semiconducting compounds containing a polycyclic unit.

US 20210070770A1 describes organic semiconducting compounds containing a polycyclic unit.

### SUMMARY

The present disclosure provides a compound of formula (I):

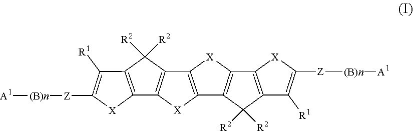

wherein: each X is independently O or S; R¹ in each occurrence is independently H or a substituent; R² in each occurrence is independently a substituent; B in each occurrence is independently a bridging unit; n in each occurrence is independently 0 or 1; A¹ in each occurrence is independently an electron-accepting group; Z is a direct bond or, together with R1, forms a monocyclic or fused ring; and the compound of formula (I) is substituted with at least one substituent of formula (II):

-(L)p-[(OCH₂CH₂)r-R³]q (II)

wherein: L is a linking group; p in each occurrence is independently is 0 or 1; r in each occurrence is independently an integer from 1 to 10; optionally 1 to 5; R³ is H or a substituent; and q is 1 if p is O and q is at least 1 if p is 1.

In some embodiments, at least one of the at least one substituents of formula (II), p is 1 and L is a C₁₋₂₀ alkylene group, optionally a C₁ or C₂ alkylene group.

In some embodiments, at least one of the at least one substituents of formula (II), p is 0.

In some embodiments, at least one of the at least one substituents of formula (II), q is 1.

In some embodiments, at least one of the at least one substituents of formula (II), R³ is H, a C₁₋₅ alkyl group, a C₁₋₅ alkoxy group or an amine group; optionally H, a C₁ alkyl group, a C₂ alkyl group, or a C₃ alkyl group.

In some embodiments, at least one R² is a substituent of formula (II).

In some embodiments, at least one A¹, optionally both A¹s, are a group of formula (IXa-1):

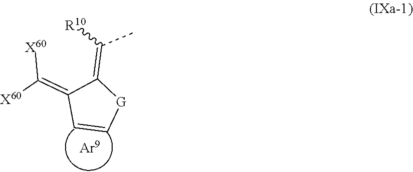

wherein:

G is C═O, C═S SO, SO₂, NR³³ or C(R³³)², wherein R³³ is CN or COOR⁴⁰ and wherein R⁴⁰ is H or a substituent; R¹⁰ is H or a substituent; Ar⁹ is an unsubstituted or substituted monocyclic or fused aromatic or heteroaromatic group; and X⁶⁰ are each independently CN, CF₃ or COOR⁴⁰ wherein R⁴⁰ in each occurrence is H or a substituent.

In some embodiments, at least one A¹, optionally both A¹s, are a group of formula (IXa-2):

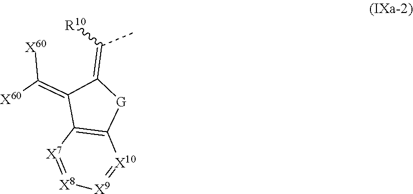

wherein: each X¹-X¹⁰ is independently CR¹² or N wherein R¹² in each occurrence is H or a substituent selected from C₁₋₂₀ hydrocarbyl and an electron withdrawing group; and R¹⁰ is H or a substituent; X⁶⁰ are each independently CN, CF₃ or COOR⁴⁰ wherein R⁴⁰ in each occurrence is H or a substituent.

In some embodiments, n is 1 and B in each occurrence is individually a vinylene, arylene, heteroarylene, arylenevinylene or heteroarylenevinylene group wherein the arylene and heteroarylene groups are monocyclic or bicyclic groups, each of which may be unsubstituted or substituted with one or more substituents.

In some embodiments, n is 1 and B in each occurrence is individually selected from units of formulae (VIa)-(VIo):

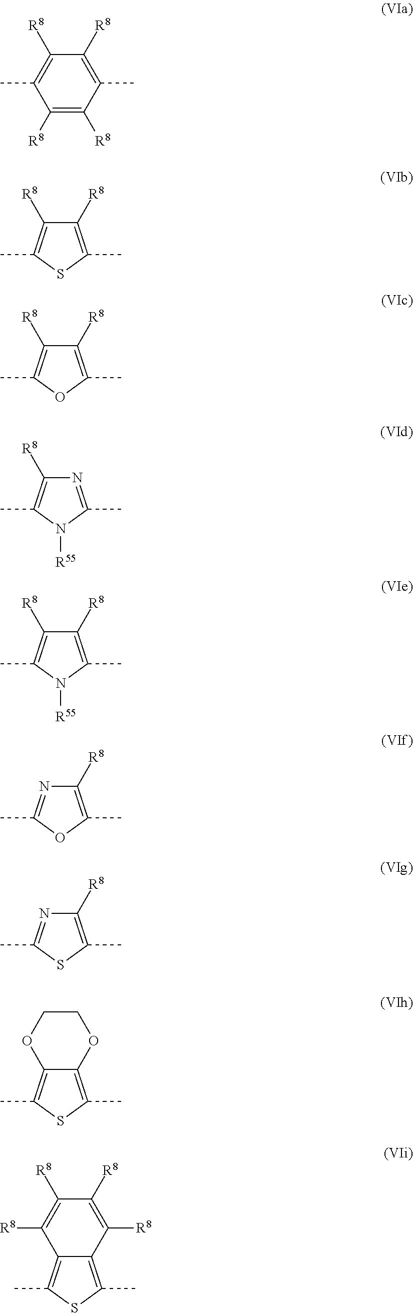

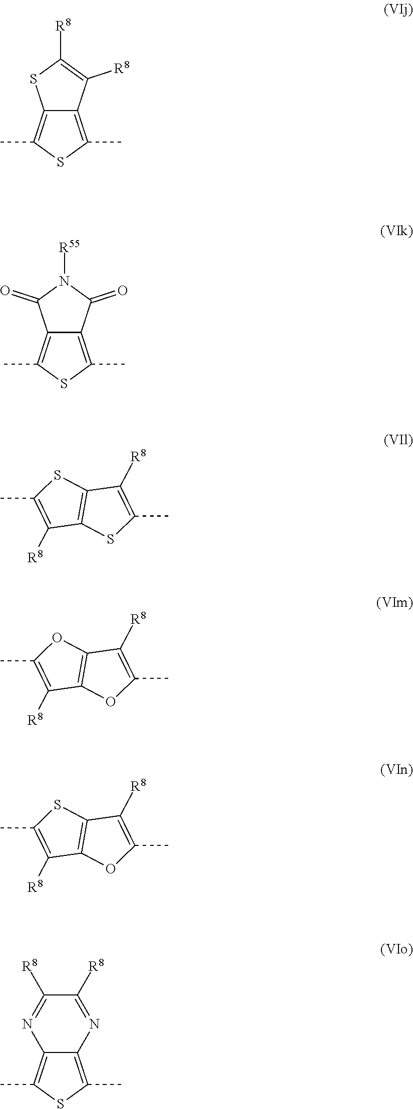

wherein:
R⁵⁵ is H or a substituent; and R& in each occurrence is independently H or a substituent.

In some embodiments, n is 1 and at least one B is substituted with a substituent of formula (II), or a substituent selected from F; CN; NO₂; C₁₋₂₀ alkyl wherein one or more non-adjacent C atoms may be replaced with O, S, NR⁶, COO or CO and one or more H atoms of the alkyl may be replaced with F; phenyl which is unsubstituted or substituted with one or more substituents; and —B(R¹⁴)₂ wherein R¹⁴ in each occurrence is a substituent.

In some embodiments, Z is a direct bond.

In some embodiments, X in each occurrence is S.

The present disclosure provides a composition comprising an electron-donating material and an electron-accepting material wherein the electron accepting material is a compound as described herein.

The present disclosure provides an organic electronic device comprising an active layer comprising a compound or composition as described herein.

Optionally, the organic electronic device is an organic photoresponsive device comprising a photoactive layer disposed between an anode and a cathode and wherein the photoactive layer comprises an electron accepting material as described herein and an electron-donating material as described herein.

Optionally, the organic photoresponsive device is an organic photodetector.

The present disclosure provides a photosensor comprising a light source and an organic photodetector as described herein wherein the organic photodetector is configured to detect light emitted from the light source.

Optionally, the light source emits light having a peak wavelength of greater than 900 nm.

The present disclosure provides a formulation comprising a compound or composition as described herein dissolved or dispersed in one or more solvents.

The present disclosure provides a method of forming an organic electronic device as described herein wherein formation of the active layer comprises deposition of a formulation as described herein onto a surface and evaporation of the one or more solvents.

It has been found that the compounds described herein (comprising a substituent of formula (II)) lead to a reduction in dark current when used in an organic photoresponsive device. Furthermore, compounds as described herein (comprising a substituent of formula (II)) show improved guest assisted molecular organisation and as such are particularly useful in forming bilayer devices.

### DESCRIPTION OF DRAWINGS

The disclosed technology and accompanying figures describe some implementations of the disclosed technology.

FIG. 1 illustrates an organic photoresponsive device according to some embodiments.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise.” “comprising.” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present disclosure provides a compound of formula (I):

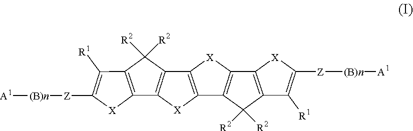

wherein: each X is independently O or S; R¹ in each occurrence is independently H a substituent; R² in each occurrence is independently a substituent; B in each occurrence is independently a bridging unit; n in each occurrence is independently 0 or 1; A¹ in each occurrence is independently an electron-accepting group; Z is a direct bond or, together with R¹, forms a monocyclic or fused ring; and the compound of formula (I) is substituted with at least one substituent of formula (II):

-(L)p-[(OCH₂CH₂)r-R³]q (II)

wherein: L is a linking group; p in each occurrence is independently is 0 or 1; r in each occurrence is independently an integer from 1 to 10; R³ is H or a substituent; and q is 1 if p is 0 and q is at least 1 if p is 1.

In some embodiments, X is S.

Each of the electron-accepting groups A¹ may have a lowest unoccupied molecular orbital (LUMO) level that is deeper (i.e., further from vacuum) than the LUMO of the electron-donating group represented by formula (Ia), preferably at least 1 eV deeper.

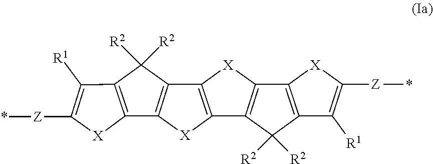

The LUMO levels of electron-accepting groups and electron-donating groups may be as determined by modelling the LUMO level of these groups, in which each bond to adjacent group is replaced with a bond to a hydrogen atom. Modelling may be performed using Gaussian09 software available from Gaussian using Gaussian09 with B3LYP (functional) and LACVP* (Basis set).

The compound of formula (I) is substituted with at least one substituent of formula (II):

-(L)p-[(OCH₂CH₂)r-R³]q (II)

In some embodiments, the compound of formula (I) is substituted with one, two, three, four, five or six substituents each individually represented by formula (II). Optionally, the compound of formula (I) is substituted with one, two, three, or four substituents each individually represented by formula (II).

In some embodiments, in at least one of the at least one substituents of formula (II), p is 1 and L is a C₆₋₂₀ arylene group, a C₅₋₂₀ heteroarylene group, or a C₁₋₂₀ alkylene group, optionally a C₆₋₁₀ arylene group or a C₁ or C₂ alkylene group. For example, in each of the at least one substituents of formula (II), p is 1 and L is a C₁₋₂₀ alkylene group, optionally a C₁ or C₂ alkylene group. For example, in each of the at least one substituents of formula (II), p is 1 and L is a C₆₋₂₀ arylene group, optionally a C₆₋₁₀ arylene group (such as phenylene).

Optionally, the compound of formula (I) is substituted with one, two, three, four, five or six substituents each independently represented by formula (II); wherein p in each occurrence is 1 and L in each occurrence is independently a C₆₋₂₀ arylene group, a C₅₋₂₀ heteroarylene group, or a C₁₋₂₀ alkylene group, optionally a C₆₋₁₀ arylene group or a C₁ or C₂ alkylene group. For example, in each of the substituents of formula (II), p is 1 and L is a C₁₋₂₀ alkylene group, optionally a C₁ or C₂ alkylene group. For example, in each of the at least one substituents of formula (II), p is 1 and L is a C₆₋₂₀ arylene group, optionally a C₆₋₁₀ arylene group (such as phenylene).

Optionally, the compound of formula (I) is substituted with one, two, three or four substituents each independently represented by formula (II); wherein p in each occurrence is 1 and L in each occurrence is independently a C₆₋₂₀ arylene group, a C₅₋₂₀ heteroarylene group, or a C₁₋₂₀ alkylene group, optionally a C₆₋₁₀ arylene group or a C₁ or C₂ alkylene group. For example, in each of the substituents of formula (II), p is 1 and L is a C₁₋₂₀ alkylene group, optionally a C₁ or C₂ alkylene group. For example, in each of the at least one substituents of formula (II), p is 1 and L is a C₆₋₂₀ arylene group, optionally a C₆₋₁₀ arylene group (such as phenylene).

In some embodiments, when B is substituted with a substituent of formula (II), p is 1 and L is a C₆₋₂₀ arylene group, optionally a C₆₋₁₀ arylene group (such as phenylene).

In some embodiments, when one or more R² is a substituent of formula (II), p is 1 and L is a C₁₋₂₀ alkylene group, optionally a C₁ or C₂ alkylene group.

In some embodiments, in at least one of the at least one substituents of formula (II), p is 0. For example, in each of the at least one substituents of formula (II), p is 0.

Optionally, the compound of formula (I) is substituted with one, two, three, four, five or six substituents each independently represented by formula (II); wherein p in each occurrence is 0. Optionally, the compound of formula (I) is substituted with one, two, three, or four substituents each individually represented by formula (II); and wherein p in each occurrence is 0.

In some embodiments, in at least one of the at least one substituents of formula (II), q is 1. For example, in each of the at least one substituents of formula (II), q is 1.

Optionally, the compound of formula (I) is substituted with one, two, three, four, five or six substituents each independently represented by formula (II); wherein q in each occurrence is 1. Optionally, the compound of formula (I) is substituted with one, two, three, or four substituents each individually represented by formula (II); and wherein q in each occurrence is 1.

In formula (II), R³ is H or a substituent. In some embodiments, R³ is H or a substituent selected from the group consisting of C₁₋₅ alkyl groups, C₁₋₅ alkoxy groups, C₆₋₂₀ aryl groups, C₁₋₂₀ heteroaryl groups and amine groups. Each of the C₁₋₅ alkyl groups, C₁₋₅ alkoxy groups, C₆₋₂₀ aryl groups, C₁₋₂₀ heteroaryl groups and amine groups may independently be unsubstituted or substituted with one or more substituents.

In some embodiments, in at least one of the at least one substituents of formula (II), R³ is a substituent selected from the group consisting of C₁₋₅ alkyl groups, C₁₋₅ alkoxy groups, C₆₋₂₀ aryl groups, C₁₋₂₀ heteroaryl groups, and amine groups as described herein. In some embodiments, the amine group is selected from the group consisting of primary amines, secondary amines or tertiary amines (preferably a tertiary amines), or a salt thereof. Optionally, the amine group is trimethylamine or a salt thereof. For example, the salt may be a bromide salt.

In some embodiments, in at least one of the at least one substituents of formula (II), R³ is H, a C₁₋₅ alkyl group, or a C₁₋₅ alkoxy group. Optionally, in at least one of the at least one substituents of formula (II), R³ is H, a C₁ alkyl group, a C₂ alkyl group, or a C₃ alkyl group. For example, in each of the at least one substituents of formula (II), R³ is H, a C₁ alkyl group, a C₂ alkyl group, or a C₃ alkyl group. Optionally in at least one of the at least one substituents of formula (II), R³ is H, a C₁ alkoxy group, a C₂ alkoxy group, or a C₃ alkoxy group. For example, in each of the at least one substituents of formula (II), R³ is H, a C₁ alkoxy group, a C₂ alkoxy group, or a C₃ alkoxy group.

In formula (II), r in each occurrence is independently an integer from 1 to 10. Optionally, r in each occurrence is independently an integer from 1 to 5, from 1 to 4, from 1 to 3, or from 1 to 2. Optionally, r in each occurrence is independently an integer from 2 to 5, from 2 to 4, or from 2 to 3. Preferably, r in each occurrence is independently an integer from 2 to 3.

In some embodiments, in at least one of the at least one substituents of formula (II), r is an integer from 1 to 10. Optionally, in each of the at least one substituents of formula (II), r is an integer from 1 to 10. For example, in each of the at least one substituents of formula (II), r is an integer from 1 to 5, from 1 to 4, from 1 to 3, or from 1 to 2. Optionally, in each of the at least one substituents of formula (II), r is independently an integer from 2 to 5, from 2 to 4, or from 2 to 3. Preferably, in each of the at least one substituents of formula (II), r is an integer from 2 to 3.

R² in each occurrence is independently a substituent. In some embodiments, R² in each occurrence is independently selected from the group consisting of a substituent of formula (II); C₁₋₂₀ alkyl wherein one or more non-adjacent C atoms may be replaced with O, S, NR⁶, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁶, COO or CO and one or more H atoms of the alkyl may be replaced with F.

Each R⁶ of any NR⁶ described anywhere herein is independently selected from H; C₁₋₂₀ alkyl wherein one or more non-adjacent C atoms other than the C atom bound to N may be replaced with O, S, NR¹¹, COO or CO and one or more H atoms of the alkyl may be replaced with F;

and phenyl which is unsubstituted or substituted with one or more substituents, optionally one or more C₁₋₁₂ alkyl groups wherein one or more non-adjacent C atoms of the alkyl may be replaced with O, S, NR¹¹, COO or CO and one or more H atoms of the alkyl may be replaced with F wherein R¹¹ is H or a C₁₋₂₀ hydrocarbyl group.

In some embodiments, R² in each occurrence is independently a substituent of formula (II). In some embodiments, at least one R² is a substituent of formula (II). In some embodiments, at least one, two, three or four R²s are a substituent of formula (II). In some embodiments, at least three or four R²s are a substituent of formula (II). In some embodiments, four R²s (i.e. each R²) are a substituent of formula (II).

R¹ in each occurrence is independently H or a substituent. In some embodiments, R¹ in each occurrence is selected from H; F; C₁₋₂₀ alkyl wherein one or more non-adjacent C atoms may be replaced with O, S, NR⁶, COO or CO and one or more H atoms of the alkyl may be replaced with F; and an aromatic or heteroaromatic group Ar³ which is unsubstituted or substituted with one or more substituents. Ar³ may be individually be any aromatic or heteroaromatic Ar³ group described herein. Optionally, one or more R¹s is H. Preferably, each R¹ is H.

Z is a direct bond or, together with R¹, forms a monocyclic or fused ring. In some embodiments, each Z is a direct bond. In some embodiments, each Z together with R¹ forms a monocyclic or fused ring. Optionally, each Z together with R¹ forms a monocyclic ring comprising a thiophene or furan ring. Each of said rings may be unsubstituted or substituted with one or more substituents. Optionally, each Z together with R¹ forms a fused ring comprising thiophene or furan rings and one or more rings selected from benzene, cyclopentadiene, tetrahydropyran, tetrahydrothiopyran and piperidine rings. Each of said rings may be unsubstituted or substituted with one or more substituents.

Unless otherwise specified, any substituent described herein may be independently selected from the group consisting of C₁₋₁₂ alkyl wherein one or more non-adjacent C atoms may be replaced with O, S, NR⁶, COO or CO and one or more H atoms of the alkyl may be replaced with F; and an aromatic group, optionally phenyl, which is unsubstituted or substituted with one or more substituents selected from F and C₁₋₁₂ alkyl wherein one or more non-adjacent C atoms may be replaced with O, S, NR⁶, COO or CO; and wherein NR⁶ is as described herein.

If a C atom of an alkyl group as described anywhere herein is replaced with another atom or group, the replaced C atom may be a terminal C atom of the alkyl group or a non-terminal C-atom.

By “non-terminal C atom” of an alkyl group as used anywhere herein means a C atom other than the C atom of the methyl group at the end of an n-alkyl chain or the C atoms of the methyl groups at the ends of a branched alkyl chain.

If a terminal C atom of a group as described anywhere herein is replaced then the resulting group may be an anionic group comprising a countercation, e.g., an ammonium or metal countercation, preferably an ammonium or alkali metal cation.

A C atom of an alkyl substituent group which is replaced with another atom or group as described anywhere herein is preferably a non-terminal C atom, and the resultant substituent group is preferably non-ionic.

### Electron-Accepting Groups A¹

The monovalent acceptor groups A¹ may each independently be selected from any such units known to the skilled person. A¹ may be the same or different, preferably the same.

Exemplary monovalent acceptor groups include, without limitation, groups of formulae (IXa)-(IXq)

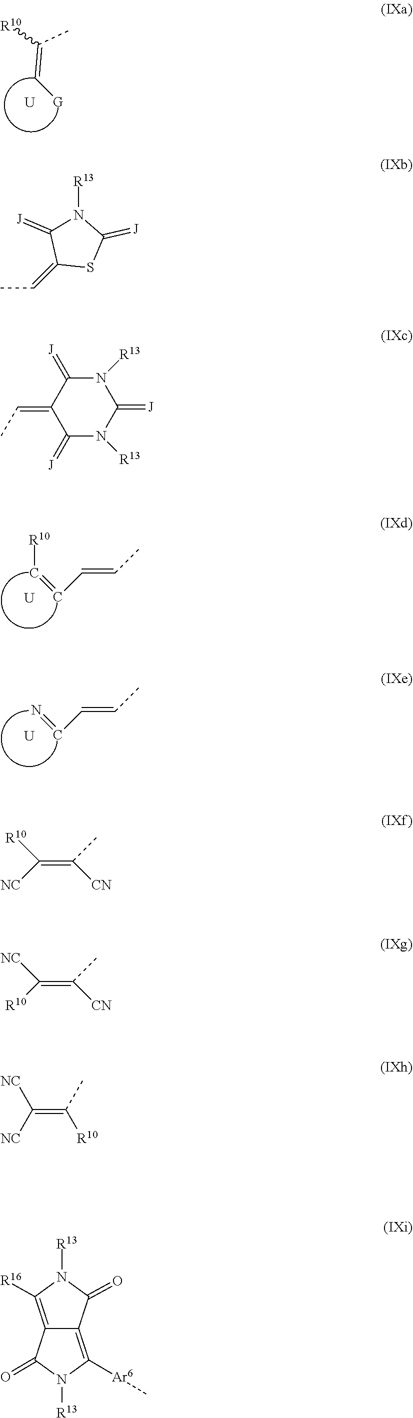

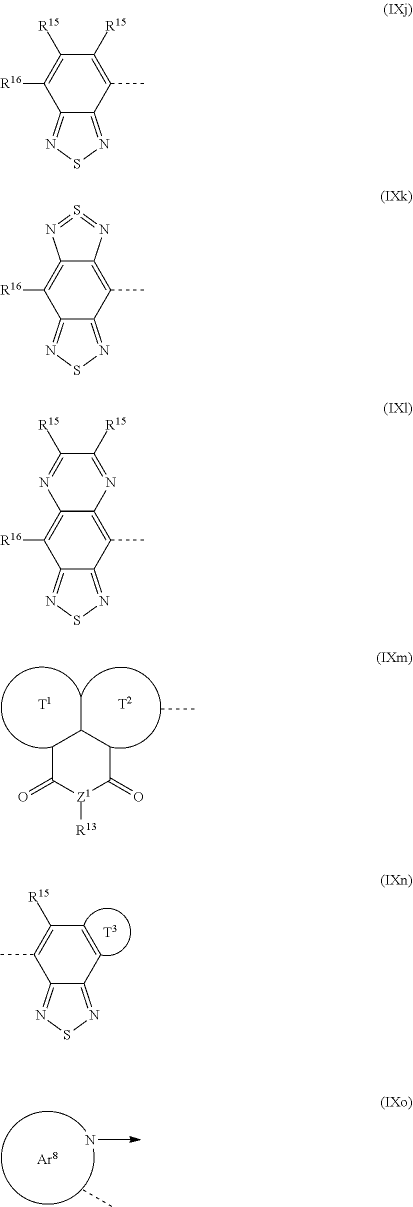

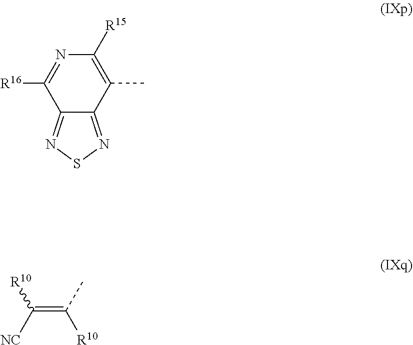

U is a 5- or 6-membered ring which is unsubstituted or substituted with one or more substituents and which may be fused to one or more further rings.

G is C═O, C═S SO, SO₂, NR³³ or C(R³³)₂ wherein R³³ is CN or COOR⁴⁰. G is preferably C═O or SO₂, more preferably C═O.

The N atom of formula (IXe) may be unsubstituted or substituted.

R¹⁰ is H or a substituent, preferably H or a substituent selected from the group consisting of C₁₋₁₂ alkyl wherein one or more non-adjacent C atoms may be replaced with O, S, NR⁶, COO or CO and one or more H atoms of the alkyl may be replaced with F; and an aromatic group, optionally phenyl, which is unsubstituted or substituted with one or more substituents selected from F and C₁₋₁₂ alkyl wherein one or more non-adjacent C atoms may be replaced with O, S, NR⁶, COO or CO.

Preferably, R¹⁰ is H.

Optionally, each R⁶ of any NR⁶ or PR⁶ described anywhere herein is independently selected from H; C₁₋₂₀ alkyl wherein one or more non-adjacent C atoms other than the C atom bound to N or P may be replaced with O, S, NR¹¹, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms of the alkyl may be replaced with O, S, NR¹¹, COO or CO and one or more H atoms of the alkyl may be replaced with F wherein R¹¹ is H or a C₁₋₂₀ hydrocarbyl group.

A C₁₋₂₀ hydrocarbyl group as described anywhere is preferably selected from C₁₋₂₀ alkyl; unsubstituted phenyl; and phenyl substituted with one or more C₁₋₁₂ alkyl groups.

J is O or S, preferably O.

R¹³ in each occurrence is a substituent, optionally C₁₋₁₂ alkyl wherein one or more non-adjacent C atoms may be replaced with O, S, NR⁶, COO or CO and one or more H atoms of the alkyl may be replaced with F.

R¹⁵ in each occurrence is independently H; F; C₁₋₁₂ alkyl wherein one or more non-adjacent C atoms may be replaced with O, S, NR⁶, COO or CO and one or more H atoms of the alkyl may be replaced with F; aromatic group Ar², optionally phenyl, which is unsubstituted or substituted with one or more substituents selected from F and C₁₋₁₂ alkyl wherein one or more non-adjacent C atoms may be replaced with O, S, NR⁶, COO or CO; or a group selected from:

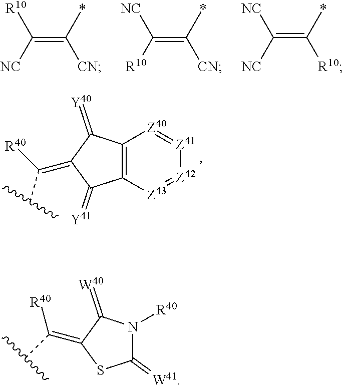

R¹⁶ is H or a substituent, preferably a substituent selected from:
—(Ar³)_{w} wherein Ar³ in each occurrence is independently an unsubstituted or substituted aryl or heteroaryl group, preferably thiophene, and w is 1, 2 or 3;

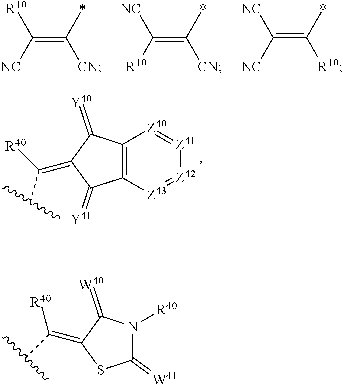

and
C₁₋₁₂ alkyl wherein one or more non-adjacent C atoms may be replaced with O, S, NR⁶, COO or CO and one or more H atoms of the alkyl may be replaced with F.

Ar⁶ is a 5-membered heteroaromatic group, preferably thiophene or furan, which is unsubstituted or substituted with one or more substituents.

Substituents of Ar³ and Ar⁶, where present, are optionally selected from C₁₋₁₂ alkyl wherein one or more non-adjacent C atoms may be replaced with O, S, NR⁶, COO or CO and one or more H atoms of the alkyl may be replaced with F.

T¹, T² and T³ each independently represent an aryl or a heteroaryl ring, optionally benzene, which may be fused to one or more further rings. Substituents of T¹, T² and T³, where present, are optionally selected from non-H groups of R²⁵. In a preferred embodiment, T³ is benzothiadiazole.

z¹ is N or P.

R²⁵ in each occurrence is independently H; F; CN; NO₂; C₁₋₁₂ alkyl wherein one or more non-adjacent C atoms may be replaced with O, S, NR⁶, COO or CO and one or more H atoms of the alkyl may be replaced with F; an aromatic group, optionally phenyl, which is unsubstituted or substituted with one or more substituents selected from F and C₁₋₁₂ alkyl wherein one or more non-adjacent C atoms may be replaced with O, S, NR⁶, COO or CO; or

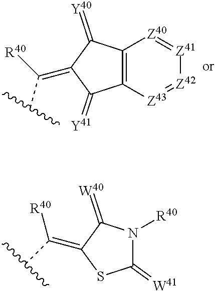

wherein Z⁴⁰, Z⁴¹, Z⁴² and Z⁴³ are each independently CR¹³ or N wherein R¹³ in each occurrence is H or a substituent, preferably a C₁₋₂₀ hydrocarbyl group;
Y⁴⁰ and Y⁴¹ are each independently O, S, NX⁷¹ wherein X⁷¹ is CN or COOR^{40;} or CX⁶⁰Z⁶¹ wherein X⁶⁰ and X⁶¹ is independently CN, CF₃ or COOR^{40;}
W⁴⁰ and W⁴¹ are each independently O, S, NX⁷¹ wherein X⁷¹ is CN or COOR^{40;} or CX⁶⁰Z⁶¹ wherein X⁶⁰ and X⁶¹ is independently CN, CF₃ or COOR⁴⁰; and
R⁴⁰ in each occurrence is H or a substituent, preferably H or a C₁₋₂₀ hydrocarbyl group.

Ar⁸ is a fused heteroaromatic group which is unsubstituted or substituted with one or more substituents, optionally one or more non-H substituents R¹⁰, and which is bound to an aromatic C atom of B and to a boron substituent of B.

Preferred groups A′ are groups having a non-aromatic carbon-carbon bond which is bound directly to the electron-donating group represented by formula (Ia) in formula (I), or, if present to B of formula (I).

Preferably at least one A¹, preferably both groups A¹, are a group of formula (IXa-1):

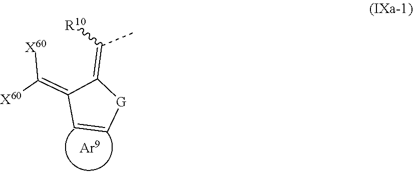

wherein:
G is as described above and is preferably C═O or SO₂, more preferably C═O;
R¹⁰ is as described above;
Ar⁹ is an unsubstituted or substituted monocyclic or fused aromatic or heteroaromatic group, preferably benzene or a monocyclic or bicyclic heteroaromatic group having C or N ring atoms only; and
X⁶⁰ are each independently CN, CF₃ or COOR⁴⁰ wherein R⁴⁰ in each occurrence is H or a substituent, preferably H or a C₁₋₂₀ hydrocarbyl group. Preferably, each X⁶⁰ is CN.

Ar⁹ may be unsubstituted or substituted with one or more substituents. Substituents of Ar⁹ are preferably selected from groups R¹² as described below.

Optionally, the group of formula (IXa-1) has formula (IXa-2):

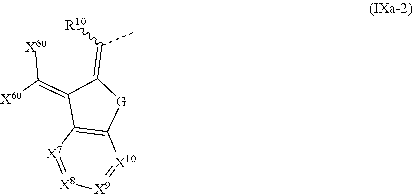

each X⁷-x10 is independently CR¹² or N wherein R¹² in each occurrence is H or a substituent selected from C₁₋₂₀ hydrocarbyl and an electron withdrawing group. Preferably, the electron withdrawing group is F, Cl, Br or CN, more preferably F, Cl or CN; and for example For CN.

The C₁₋₂₀ hydrocarbyl group R¹² may be selected from C₁₋₂₀ alkyl; unsubstituted phenyl; and phenyl substituted with one or more C₁₋₁₂ alkyl groups.

In a particularly preferred embodiment, each of X⁷-X¹⁰ is CR¹² and each R¹² is independently selected from H or an electron-withdrawing group, preferably H, F or CN. According to this embodiment, R¹² of X⁸ and X⁹ is an electron-withdrawing group, preferably F or CN.

Exemplary groups of formula (IXd) include:

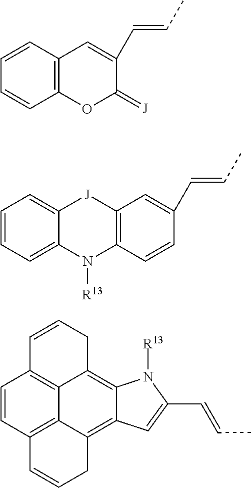

Exemplary groups of formula (IXe) include:

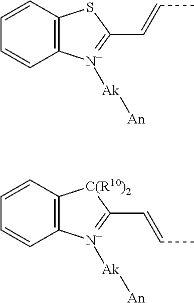

An exemplary group of formula (IXq) is:

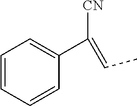

An exemplary group of formula (IXg) is:

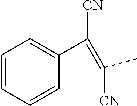

An exemplary group of formula (IXj) is:

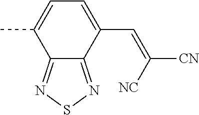

wherein Ak is a C₁₋₁₂ alkylene chain in which one or more C atoms may be replaced with O, S, NR⁶, CO or COO; An is an anion, optionally —SO₃⁻; and each benzene ring is independently unsubstituted or substituted with one or more substituents selected from substituents described with reference to R^{10.}

Exemplary groups of formula (IXm) are:

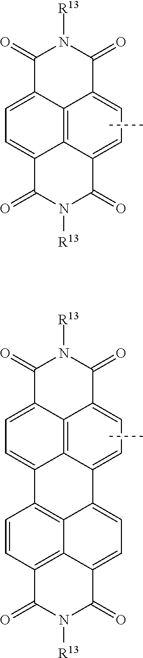

An exemplary group of formula (IXn) is:

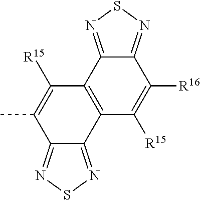

Groups of formula (IXo) are bound directly to a bridging group B substituted with a group of formula —B(R¹⁴)₂ wherein R¹⁴ in each occurrence is a substituent, optionally a C₁₋₂₀ hydrocarbyl group; → is a bond to the boron atom —B(R¹⁴)₂; and [symbol] is a C—C bond between formula (IXo) and the bridging group.

Optionally, R¹⁴ is selected from C₁₋₁₂ alkyl; unsubstituted phenyl; and phenyl substituted with one or more C₁₋₁₂ alkyl groups.

The group of formula (IXo), the B group and the B(R¹⁴)₂ substituent of B may be linked together to form a 5- or 6-membered ring.

Optionally groups of formula (IXo) are selected from:

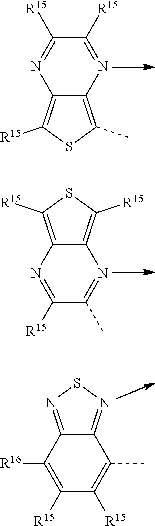

### Bridging Units

Bridging units B are preferably cach independently selected from vinylene, arylene, heteroarylene, arylenevinylene and heteroarylenevinylene wherein the arylene and heteroarylene groups are monocyclic or bicyclic groups, each of which may be unsubstituted or substituted with one or more substituents.

Optionally, cach B is independently selected from units of formulae (VIa)-(VIn):

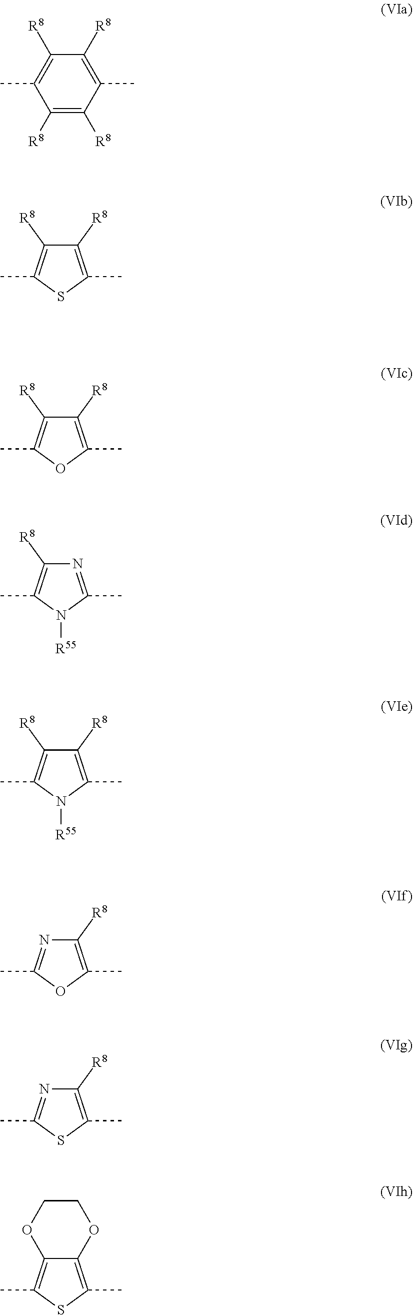

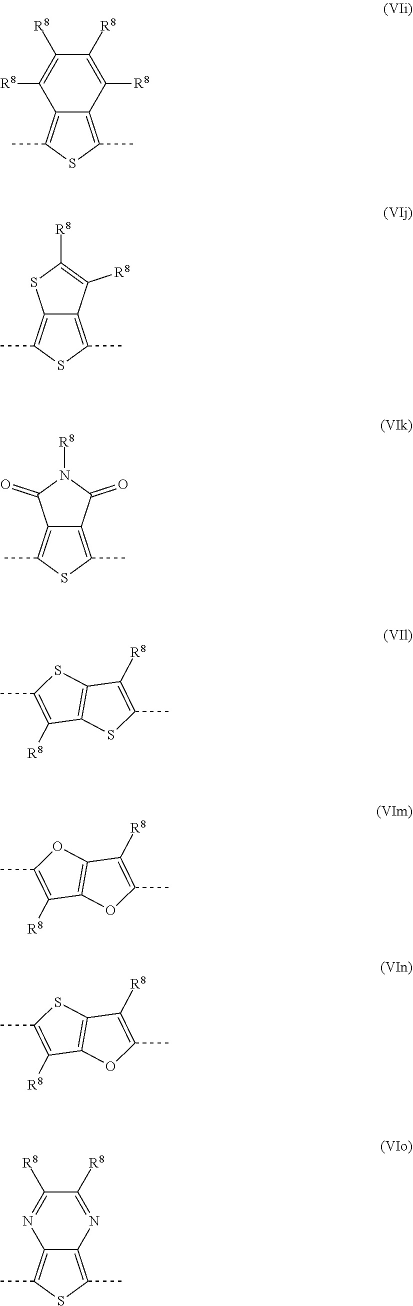

wherein R⁵⁵ is H or a substituent; R⁸ in each occurrence is independently H or a substituent, preferably H or a substituent selected from a substituent of formula (II) as described anywhere herein; F; CN; NO₂; C₁₋₂₀ alkyl wherein one or more non-adjacent C atoms may be replaced with O, S, NR⁶, COO or CO and one or more H atoms of the alkyl may be replaced with F; phenyl which is unsubstituted or substituted with one or more substituents; and —B(R¹⁴)₂ wherein R¹⁴ in each occurrence is a substituent, optionally a C₁₋₂₀ hydrocarbyl group.

R⁸ groups of formulae (VIa), (VIb) and (VIc) may be linked to form a bicyclic ring which may be substituted with one or more substituents, optionally one or more substituents selected from F; CN; NO₂; C₁₋₂₀ alkyl wherein one or more non-adjacent C atoms may be replaced with O, S, NR⁶, COO or CO and one or more H atoms of the alkyl may be replaced with F.

R⁸ is preferably H, C₁₋₂₀ alkyl or C₁₋₁₉ alkoxy. Optionally, one R⁸ is H and one R⁸ is C₁₋₂₀ alkyl or C₁₋₁₉ alkoxy.

Preferably, each B (when present) is independently a unit of formula (VIb). Optionally, at least one R⁸ is a C₁₋₁₉ alkoxy, optionally a C₁₋₁₀ alkoxy or a C₁₋₅ alkoxy. In some embodiments, at least one R⁸ is ethoxy or methoxy. Optionally, at least one R⁸ is H. Preferably, one R⁸ is H and one R⁸ is a C₁₋₁₉ alkoxy, optionally a C₁₋₁₀ alkoxy or a C₁₋₅ alkoxy (for example, ethoxy or methoxy).

In compounds of formula (I), n in each occurrence is independently 0 or 1. In some embodiments, n in each occurrence is 0. In some embodiments, n in each occurrence is 1.

In some embodiments, n is 1 and at least one B (optionally both Bs) is substituted with a substituent of formula (II), or a substituent selected from F; CN; NO₂; C₁₋₂₀ alkyl wherein one or more non-adjacent C atoms may be replaced with O, S, NR⁶, COO or CO and one or more

H atoms of the alkyl may be replaced with F: phenyl which is unsubstituted or substituted with one or more substituents; and —B(R¹⁴)₂ wherein R¹⁴ in cach occurrence is a substituent.

Exemplary compounds of formula (I) include, without limitation:

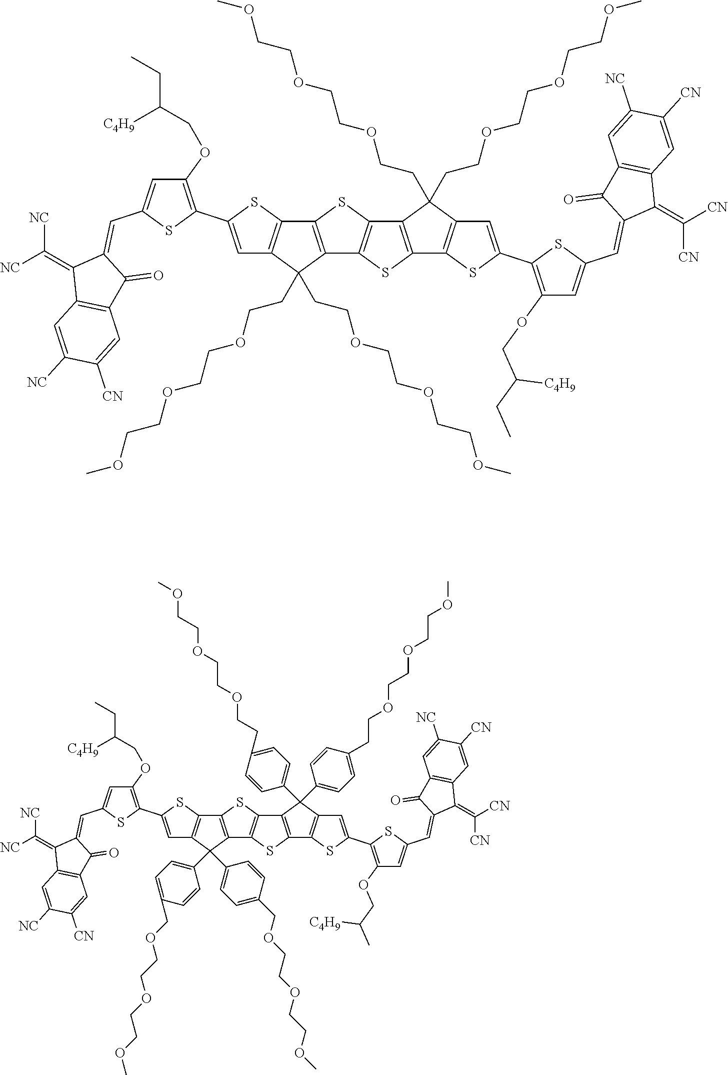

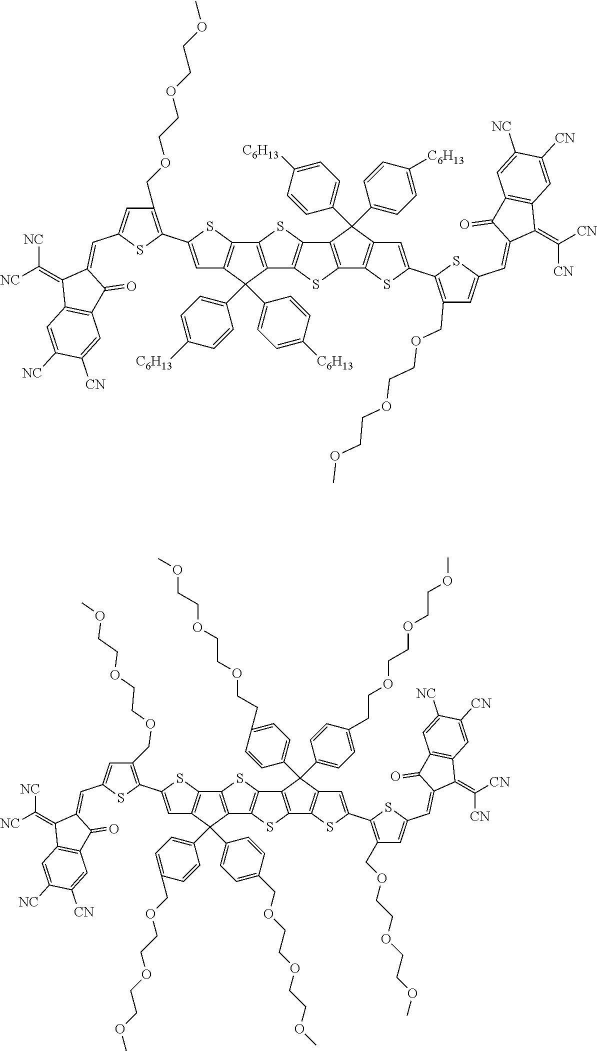

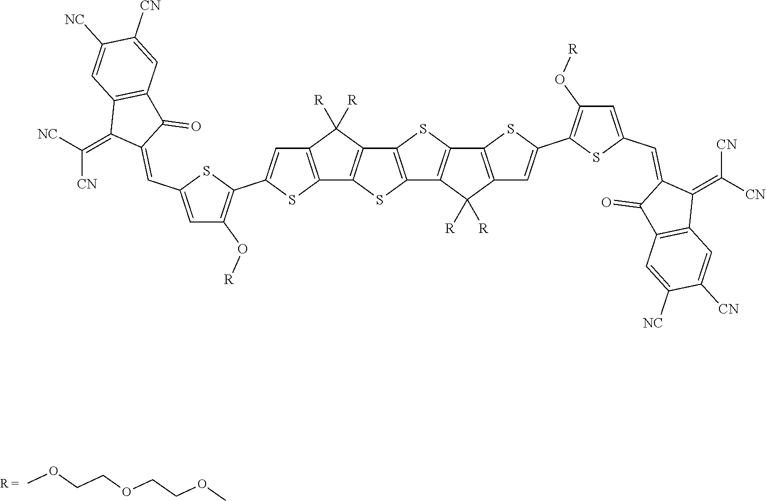

### Electron-Donating Material

Exemplary donor materials are disclosed in, for example, WO2013/051676, the contents of which are incorporated herein by reference.

The electron-donating material may be a non-polymeric or polymeric material.

In a preferred embodiment the electron-donating material is an organic conjugated polymer, which can be a homopolymer or copolymer including alternating, random or block copolymers. The conjugated polymer is preferably a donor-acceptor polymer comprising alternating electron-donating repeat units and electron-accepting repeat units.

Preferred are non-crystalline or semi- crystalline conjugated organic polymers.

Further preferably the electron-donating polymer is a conjugated organic polymer with a low bandgap, typically between 2.5 eV and 1.5 eV, preferably between 2.3 eV and 1.8 eV.

Optionally, the electron-donating polymer has a HOMO level no more than 5.5 eV from vacuum level. Optionally, the electron-donating polymer has a HOMO level at least 4.1 eV from vacuum level. As exemplary electron-donating polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4-bisubstituted selenophene), poly(bisthiophene), poly(terthiophene), poly(bisselenophene), poly(terselenophene), polythieno[2,3-b]thiophene, polythieno[3,2-b]thiophene, polybenzothiophene, polybenzo[1,2-b:4,5-b′]dithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned.

Preferred examples of donor polymers are copolymers of polyfluorenes and polythiophenes, each of which may be substituted, and polymers comprising benzothiadiazole-based and thiophene-based repeating units, each of which may be substituted.

A particularly preferred donor polymer comprises donor unit (VIIa) provided as a repeat unit of the polymer, most preferably with an electron-accepting repeat unit, for example divalent electron-accepting units A¹ as described herein provided as polymeric repeat units.

Another particularly preferred donor polymer comprises repeat units of formula (X):

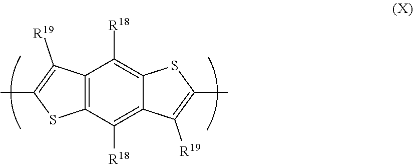

wherein R¹⁸ and R¹⁹ are each independently selected from H; F; C₁₋₁₂ alkyl wherein one or more non-adjacent, non-terminal C atoms may be replaced with O, S, COO or CO and one or more H atoms of the alkyl may be replaced with F; or an aromatic or heteroaromatic group Ar⁶ which is unsubstituted or substituted with one or more substituents selected from F and C₁₋₁₂ alkyl wherein one or more non-adjacent, non-terminal C atoms may be replaced with O, S, COO or CO.

The donor polymer is preferably a donor-acceptor (DA) copolymer comprising a donor repeat unit, for example a repeat unit of formula (VIIa) or (X), and an acceptor repeat unit.

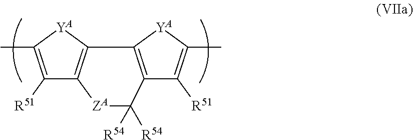

wherein Y^{A} in each occurrence is independently O, S or NR⁵⁵; Z^{A} in each occurrence is O, CO, S, NR⁵⁵ or C(R⁵⁴)₂; and R⁵¹, R⁵⁴ and R⁵⁵ independently in each occurrence is H or a substituent.

Optionally, R⁵¹ independently in each occurrence is selected from H; F; C₁₋₂₀ alkyl wherein one or more non-adjacent C atoms may be replaced with O, S, NR⁶, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g., phenyl.

Preferably, each R⁵⁴ is selected from the group consisting of:

### H;

### F;

linear, branched or cyclic C₁₋₂₀ alkyl wherein one or more non-adjacent C atoms may be replaced by O, S, NR¹⁷, CO or COO wherein R¹⁷ is a C₁₋₁₂ hydrocarbyl and one or more H atoms of the C₁₋₂₀ alkyl may be replaced with F; and
a group of formula (Ak)u-(Ar⁷)v wherein Ak is a C₁₋₂₀ alkylene chain in which one or more non-adjacent C atoms may be replaced with O, S, NR⁶, CO or COO; u is 0 or 1; Ar⁷ in each occurrence is independently an aromatic or heteroaromatic group which is unsubstituted or substituted with one or more substituents; and v is at least 1, optionally 1, 2 or 3.

Preferably, each R⁵¹ is H.

Preferably, R⁵⁵ as described anywhere herein is H or C₁₋₃₀ hydrocarbyl group.

Exemplary groups of formula (VIIa) include, without limitation:

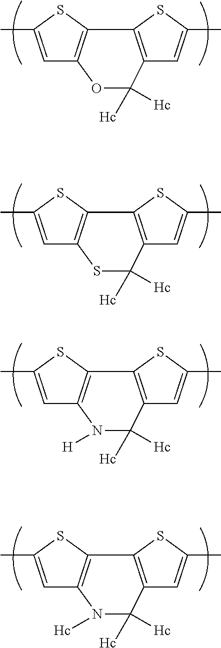

wherein Hc in each occurrence is independently a C₁₋₂₀ hydrocarbyl group, e.g., C₁₋₂₀ alkyl, unsubstituted aryl, or aryl substituted with one or more C₁₋₁₂ alkyl groups. The aryl group is preferably phenyl.

### Organic Electronic Device

A compound of formula (I) may be provided as an active layer of an organic electronic device. In a preferred embodiment, a photoactive layer of an organic photoresponsive device, more preferably an organic photodetector, comprises a compound or composition as described herein.

The photoactive layer as described herein comprises an electron-accepting material and an electron-donating material. In some embodiments, the photoactive layer is a single bulk heterojunction layer containing both an electron-accepting material and an electron-donating material. In other embodiments, the photoactive layer comprises two or more sublayers.

In some embodiments, the photoactive layer comprises a bilayer comprising an electron-accepting sub-layer comprising or consisting of an electron-accepting compound as described herein and an electron-donating sub-layer comprising or consisting of an electron donating material wherein the sub-layers are directly adjacent to and in contact with one another.

In yet further embodiments, the photoactive layer comprises a bulk heterojunction sub-layer containing both an electron-accepting material and an electron-donating material, and one or both of an electron-accepting sub-layer comprising an electron-accepting material on a cathode side of the bulk heterojunction sub-layer and an electron-donating sub-layer comprising an electron-donating material on an anode side of the bulk heterojunction sub-layer.

A bulk heterojunction layer as described herein comprises or consists of an electron-donating material and an electron-accepting compound of formula (I) as described herein.

In some embodiments, the bulk heterojunction layer contains two or more accepting materials and/or two or more electron-accepting materials.

In some embodiments, the weight of the electron-donating material(s) to the electron-accepting material(s) is from about 1:0.5 to about 1:2, preferably about 1:1.1 to about 1:2.

Preferably, the electron-donating material has a type II interface with the electron-accepting material, i.e. the electron-donating material has a shallower HOMO and LUMO that the corresponding HOMO and LUMO levels of the electron-accepting material. Preferably, the compound of formula (I) has a HOMO level that is at least 0.05 eV deeper, optionally at least 0.10 eV deeper, than the HOMO of the electron-donating material.

Optionally, the gap between the HOMO level of the electron-donating material and the LUMO level of the electron-accepting compound of formula (I) or (II) is less than 1.4 eV.

Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml) and spun at 3000 rpm directly on to the glassy carbon working electrode.

LUMO=4.8-E ferrocene (peak to peak average)−E reduction of sample (peak maximum).

HOMO=4.8-E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data.

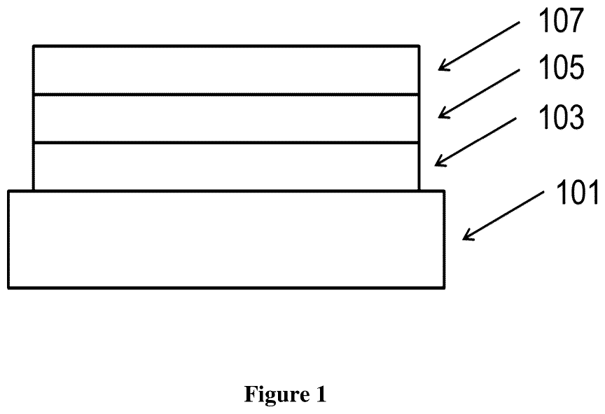

FIG. 1 illustrates an organic photoresponsive device according to some embodiments of the present disclosure. The organic photoresponsive device comprises a cathode 103, an anode 107 and a photoactive bulk heterojunction layer 105 disposed between the anode and the cathode. The organic photoresponsive device may be supported on a substrate 101, optionally a glass or plastic substrate.

The organic photoresponsive device illustrated in FIG. 1 comprises a bulk heterojunction layer. In other embodiments, the photoactive layer comprises or consists of an electron-accepting sub-layer and an electron-donating sub-layer.

Each of the anode and cathode may independently be a single conductive layer or may comprise a plurality of layers.

At least one of the anode and cathode is transparent so that light incident on the device may reach the photoactive layer. In some embodiments, both of the anode and cathode are transparent. The transmittance of a transparent electrode may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and photoactive layer shown in FIG. 1. In some embodiments, a hole-transporting layer is disposed between the anode and the photoactive layer. In some embodiments, an electron-transporting layer is disposed between the cathode and the photoactive layer. In some embodiments, a work function modification layer is disposed between the photoactive layer and the anode, and/or between the photoactive layer and the cathode.

The area of the OPD may be less than about 3 cm², less than about 2 cm², less than about 1 cm², less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The photoactive layer contains an electron-accepting compound as described herein and an electron-donating material. A bulk heterojunction layer may consist of these materials or may comprise one or more further materials, for example one or more further electron-donating materials and/or one or more further electron-accepting compounds.

### Fullerene

In some embodiments, a compound of formula (I) is the only electron-accepting material of an electron-accepting sub-layer or a bulk heterojunction layer as described herein.

In some embodiments, an electron-accepting sub-layer or a bulk heterojunction layer as described herein contains a compound of formula (I) and one or more further electron-accepting materials. Preferred further electron-accepting materials are fullerenes. The compound of formula (I) : fullerene acceptor weight ratio may be in the range of about 1:0.1-1:1, preferably in the range of about 1:0.1-1:0.5.

Fullerenes may be selected from, without limitation, C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g. tolyl-C₆₁-butyric acid methyl ester (C₆₀TCBM)), and ThCBM-type fullerene derivatives (e.g. thienyl-C₆₁-butyric acid methyl ester (C₆₀ThCBM).

Fullerene derivatives may have formula (V):

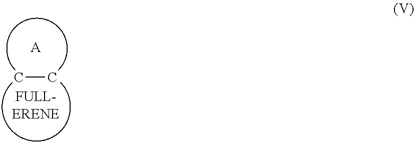

wherein A, together with the C-C group of the fullerene, forms a monocyclic or fused ring group which may be unsubstituted or substituted with one or more substituents.

Exemplary fullerene derivatives include formulae (Va), (Vb) and (Vc):

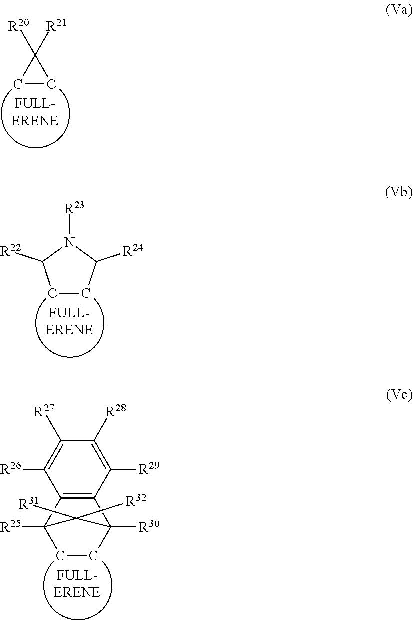

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent C atoms may be replaced with O, S, NR⁶, CO or COO and one or more H atoms may be replaced with F.

Substituents of aryl or heteroaryl, where present, are optionally selected from C₁₋₁₂ alkyl wherein one or more non-adjacent C atoms may be replaced with O, S, NR⁶, CO or COO and one or more H atoms may be replaced with F.

### Formulations

Formation of an electron-accepting sub-layer of a photoactive layer or a photoactive bulk heterojunction layer as described herein preferably comprises deposition of a formulation comprising electron-accepting material(s) including the compound of formula (I) and any other components of the electron-accepting sub-layer or bulk heterojunction layer dissolved or dispersed in a solvent or a mixture of two or more solvents. It will be understood that in the case of a bulk heterojunction layer the formulation further comprises one or more electron-donating materials.

The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

In some embodiments, formation of an organic photoactive device comprises, in any order, deposition of a first formulation comprising the compound of formula (I) and a solvent or solvent mixture comprising or consisting of a polar solvent and evaporation of the or each solvent of the first formulation; and deposition of a second formulation comprising an electron-donating material for forming an electron-donating sub-layer or a charge-transporting or charge-blocking material for forming a charge-transporting or charge-blocking layer, dissolved in one or more non-polar solvents and evaporation of the or each solvent of the second formulation.

In the case where the second formulation is deposited onto a layer or sub-layer formed from the first formulation, the layer or sub-layer containing the compound of formula (I) is suitably not dissolved by the second formulation comprising one or more non-polar solvents.

In the case where the first formulation is deposited onto a layer or sub-layer formed from the second formulation, the layer or sub-layer containing the non-fluorinated active organic material is suitably not dissolved by the halogenated solvent of the first formulation in the case where the first formulation is deposited onto this layer.

The solvent or solvents of a first formulation comprising a compound of formula (I) preferably includes at least one polar solvent. Polar solvents may be protic or aprotic. Exemplary protic solvents are C₁₋₁₂ alkyl alcohols and diols wherein one or more non-adjacent, non-terminal C atoms, other than a C atom bound to OH, may be replaced with O and wherein one or more H atoms may be replaced with F, for example methanol, ethanol, propanol, butoxyethanol, ethylene glycol, 1-methoxy-2-propanol and monofluoro-, polyfluoro- or perfluoro-alcohols. Exemplary aprotic polar solvents have a dielectric constant at 20° C. of at least 20.

The one or more solvents of the second formulation may optionally comprise or consist of benzene or naphthalene substituted with one or more substituents selected from fluorine, chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The second formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulations as described herein may comprise further components in addition to the electron-accepting material of formula (I), the one or more solvents and, in the case of a formulation of forming a bulk heterojunction layer, an electron-donating material. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

### Applications

A circuit may comprise the OPD connected to one or more of a voltage source for applying a reverse bias to the device; a device configured to measure photocurrent; and an amplifier configured to amplify an output signal of the OPD. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm or at least 1000 nm, optionally in the range of 900-1500 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g., due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

### Synthesis of Example Compound 1

Example Compound 1 was synthesised according to Scheme 1.

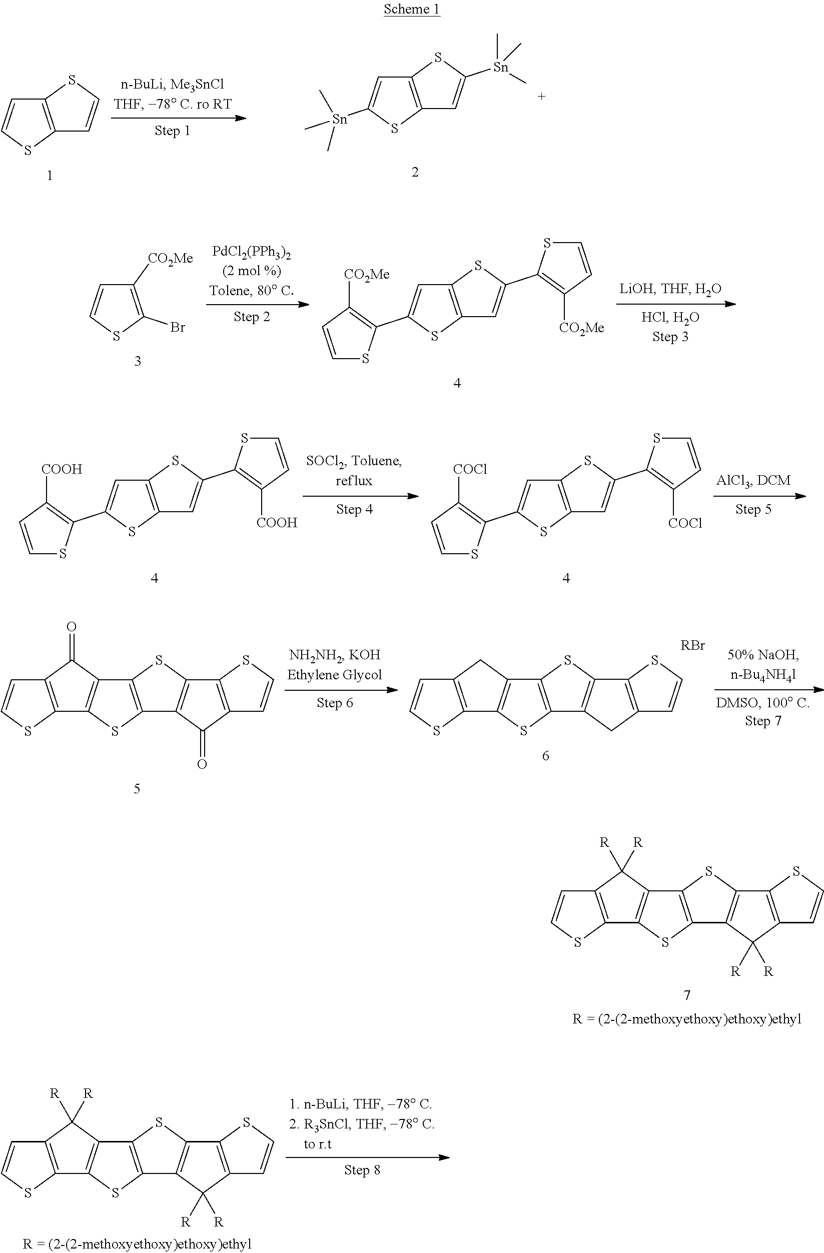

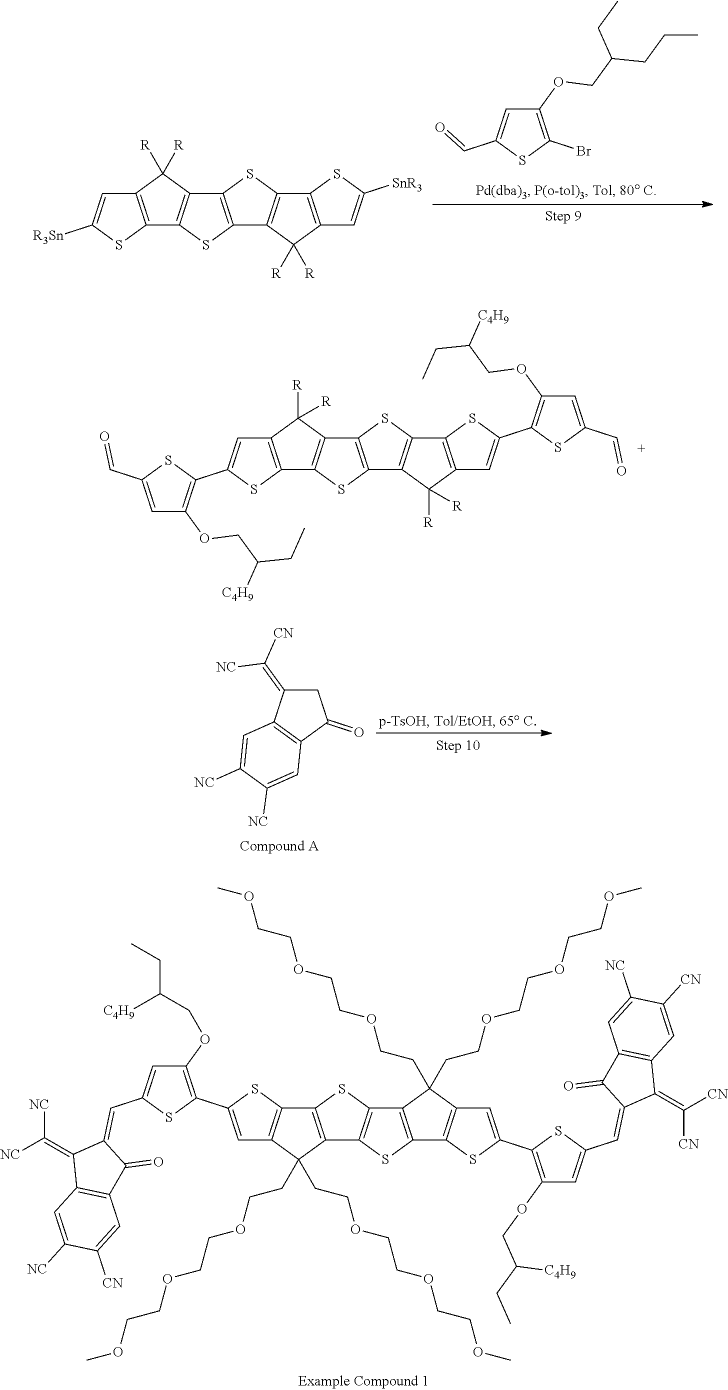

Compound A was synthesised as described in WO 2022/129137. which is incorporated in its entirety herein.

1. A compound of formula (I): 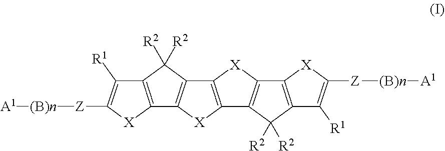
wherein:
each X is independently O or S;
R¹ in each occurrence is independently H or a substituent;
R² in each occurrence is independently a substituent;
B in each occurrence is independently a bridging unit;
n in each occurrence is independently 0 or 1;
A¹ in each occurrence is independently an electron-accepting group;
Z is a direct bond or, together with R¹, forms a monocyclic or fused ring; and
the compound of formula (I) is substituted with at least one substituent of formula (II):
-(L)p-[(OCH₂CH₂)r-R³]q (II)
wherein:
L is a linking group;
p in each occurrence is independently 0 or 1;
r in each occurrence is independently an integer from 1 to 10; optionally 1 to 5;
R³ is H or a substituent; and
q is 1 if p is 0 and q is at least 1 if p is 1.

2. A compound according to claim 1, wherein in at least one of the at least one substituents of formula (II), p is 1 and L is a C₁₋₂₀ alkylene group, optionally a C₁ or C₂ alkylene group.

3. A compound according to claim 1, wherein in at least one of the at least one substituents of formula (II), p is 0.

4. A compound according to claim 1, wherein in at least one of the at least one substituents of formula (II), q is 1.

5. A compound according to claim 1, wherein in at least one of the at least one substituents of formula (II), R³ is H, a C₁₋₅ alkyl group, a C₁₋₅ alkoxy group or an amine group;
optionally H, a C₁ alkyl group, a C₂ alkyl group, or a C₃ alkyl group.

6. A compound according to claim 1, wherein at least one R² is a substituent of formula (II).

7. A compound according to claim 1, wherein at least one A¹, optionally both A¹s, are a group of formula (IXa-1): 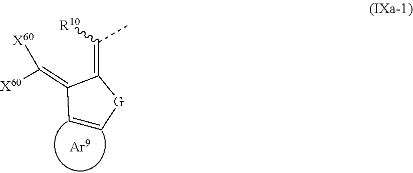
wherein:
G is C═O, C═S SO, SO₂, NR³³ or C(R³³)₂, wherein R³³ is CN or COOR⁴⁰ and wherein
R⁴⁰ is H or a substituent;
R¹⁰ is H or a substituent;
Ar⁹ is an unsubstituted or substituted monocyclic or fused aromatic or heteroaromatic group; and
X⁶⁰ are each independently CN, CF₃ or COOR⁴⁰ wherein R⁴⁰ in each occurrence is H or a substituent.

8. A compound according to claim 1, wherein at least one A¹, optionally both A¹s, are a group of formula (IXa-2): 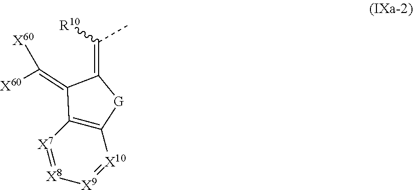
wherein:
each X⁷-X¹⁰ is independently CR¹² or N wherein R¹² in each occurrence is H or a substituent selected from C₁₋₂₀ hydrocarbyl and an electron withdrawing group; and
R¹⁰ is H or a substituent;
X⁶⁰ are each independently CN, CF₃ or COOR⁴⁰ wherein R⁴⁰ in each occurrence is H or a substituent.

9. A compound according to claim 1, wherein n is 1 and B in each occurrence is individually a vinylene, arylene, heteroarylene, arylenevinylene or heteroarylenevinylene group wherein the arylene and heteroarylene groups are monocyclic or bicyclic groups, each of which may be unsubstituted or substituted with one or more substituents.

10. A compound according to claim 1, wherein n is 1 and B in each occurrence is individually selected from units of formulae (VIa)-(VIn): 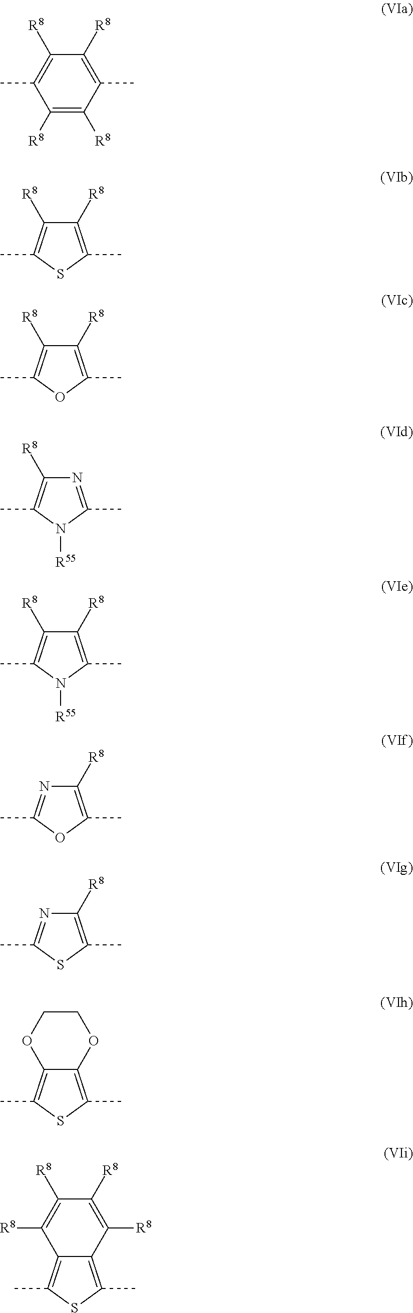 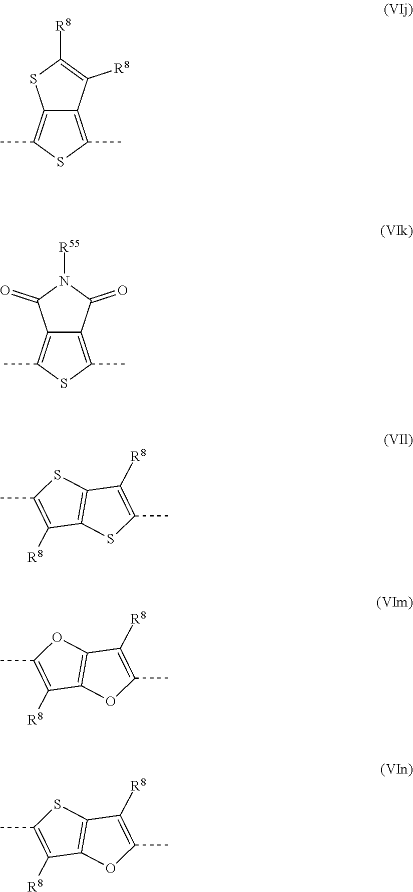
wherein:
R⁵⁵ is H or a substituent; and
R⁸ in each occurrence is independently H or a substituent.

11. A compound according to claim 1, wherein n is 1 and at least one B is substituted with a substituent of formula (II), or a substituent selected from F; CN; NO₂; C₁₋₂₀ alkyl wherein one or more non-adjacent C atoms may be replaced with O, S, NR⁶, COO or CO and one or more H atoms of the alkyl may be replaced with F; phenyl which is unsubstituted or substituted with one or more substituents; and —B(R¹⁴)₂ wherein R¹⁴ in each occurrence is a substituent.

12. A compound according to claim 1, wherein Z is a direct bond.

13. A compound according to claim 1, wherein X in each occurrence is S.

14. A composition comprising an electron-donating material and an electron-accepting material wherein the electron accepting material is a compound according to claim 1.

15. An organic electronic device comprising an active layer comprising a compound according to claim lor a composition according to claim 14.

16. An organic electronic device according to claim 15 wherein the organic electronic device is an organic photoresponsive device and the active layer is a photoactive layer disposed between an anode and a cathode of the organic photoresponsive device.

17. An organic electronic device according to claim 16 wherein the organic photoresponsive device is an organic photodetector.

18. A photosensor comprising a light source and an organic photodetector according to claim 17 wherein the photosensor is configured to detect light emitted from the light source.

19. The photosensor according to claim 18, wherein the light source emits light having a peak wavelength of greater than 900 nm.

20. A formulation comprising a compound according to any one of claims 1 or a composition according to claim 14 dissolved or dispersed in one or more solvents.
